(19) United States
(12) Patent Application Publication
(10) Pub. No.: US 2010/0136669 A1
(43) Pub. Date: Jun. 3, 2010

(54) MICROCHIP WITH ACCESSIBLE FRONT SIDE

(75) Inventors: Johannes Wilhelmus Weekamp, Beek En Donk (NL); Menno Willem Jose Prins, Rosmalen (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/063,241

(22) PCT Filed: Aug. 1, 2006
(86) PCT No.: PCT/IB06/52624

§ 371 Date: Feb. 8, 2008

(30) Foreign Application Priority Data
Aug. 9, 2005 (EP) 05107309.6

(51) Int. Cl. C12M 1/34; H05K 1/00; H05K 3/30

(52) U.S. Cl. 435/287.2; 361/760; 29/832

(57) ABSTRACT

The invention relates to a microchip assembly (40) that is particularly suited for biosensor applications in which a sensitive front side (13) of the associated microchip (10) has to be brought close to a sample. The microchip (10) comprises grooves or recesses on the front side of its substrate in which the connections to electrical tracks (42) are located. Thus the interconnection plane of the assembly is shifted backwards with respect to the sensitive front side (13) of the microchip (10), allowing a free access to the latter.

The invention relates to a microchip with processing circuits in the front side of a substrate. Moreover, it is related to a microchip assembly and a microfluidic device comprising such a microchip, and to a process for the production of said microchip assembly.

From the WO 2005/010543 A1 and WO 2005/010542 A2 a microchip is known which may for example be used in a microfluidic biosensor for the detection of biological molecules labeled with magnetic beads. The sensor chip is provided with coupling circuits comprising wires for the generation of a magnetic field and Giant Magneto Resistances (GMR) for the detection of stray fields generated by magnetized beads. The coupling circuits are fabricated at a “sensitive side” of the chip on a semiconductor substrate, and each sensor chip is attached behind a hole in the wall of a microfluidic channel with its sensitive side facing the channel. A disadvantage of these known devices is that the sample fluid has to dive into a recess to reach the sensitive chip surface. This may create regions of low or stagnant flow and generally impairs the measurement.

Based on this situation it was an object of the present invention to provide means that particularly allow the construction of an improved microfluidic device of the kind described above.

This object is achieved by a microchip according to claim 1, by a microchip assembly according to claim 7, by a microfluidic device according to claim 8, and by a process according to claim 9. Preferred embodiments are disclosed in the dependent claims.

According to its first aspect, the invention relates to a microelectronic chip or “microchip” comprising the following components:
a) a substrate;
b) processing circuits being located in a side (i.e. a substantially planar piece of the surface) of said substrate, wherein said side is called the “front side” of the substrate or microchip in the following;
c) at least one recess in said front side of the substrate;
d) at least one terminal pad for electrical connections to the processing circuits, the pad being located in the aforementioned recess.

The substrate on which the processing circuits are disposed or fabricated may particularly be one of the known semiconductor materials (like silicon Si or GaAs), glass, ceramic, and organic material or mixtures thereof. Typically, there is an intimate contact and junction between substrate and processing circuits, with the circuits for example being generated by doping in the surface layers of the substrate and/or by deposition of material on said surface.

As the terminal pads of the microchip are located in recesses, the corresponding connections to external lines or wires are shifted rearwards with respect to the front side of the substrate. The processing circuits in said front side therefore become better accessible, which makes the microchip suited for applications where the processing circuits are to be brought into the vicinity of another object.

The dimensions of the at least one recess on the microchip in connection with the intended bonding technique (for example wire bonding) determine how well the processing circuits will be accessible. According to a preferred embodiment, the at least one recess has a depth of more than 20 μm, preferably more than 30 μm (measured with respect to the front side of the substrate). Moreover, the at least one recess has preferably a width ranging from 100 to 1000 μm, most preferably from 200 to 400 μm. The recess may particularly be disposed at the border of the substrate, thus forming a stepped edge of the microchip.

According to a preferred embodiment, bumps (i.e. a raised contact-pads) are disposed on the terminal pads. As known in the state of the art, bumps may consist of a metal like gold or copper or a soldering material to which electrical tracks or wires can be bonded. Due to their arrangement in the recesses, the bumps extend correspondingly less far in forward direction of the microchip as they normally would. Preferably, the bumps are even completely retracted from the front side, i.e. they do not project beyond the front side. In this case it is possible to produce electrical connections to the bumps that do not extend beyond the front side either, thus providing a freely accessible, exposed position of the processing circuits in the front side.

The processing circuits may in principle have any design and may serve any purpose. Preferably, the processing circuits comprise coupling circuits that are adapted to perform and process a wireless physical interaction. Said physical interaction may particularly comprise the generation and/or detection of electromagnetic fields. It may however also involve other physical phenomena (e.g. thermal conduction). Typically, these interactions are limited to short distances in the order of the extensions of the microchip, particularly in the order the thickness of the chip or its components, which may range from zero up to 100 μm, preferably up to 10 μm. It should be noted that the coupling circuits are also capable to process the physical interactions. This shall quite generally mean that they have a controllable influence on these interactions and/or that they are influenced by the interactions in a controllable way. This distinguishes the coupling circuits from usual circuits of a microchip, which are of course also subject to physical interactions, but wherein said interactions are only (undesired) interferences and effectively without influence on the normal processing function of the circuits. In contrast to this, the coupling circuits are particularly designed to exploit the experienced wireless physical interactions.

The coupling circuits may particularly be designed in such a way that they implement a sensor, preferably a capacitive sensor, a light sensor, an electrical current sensor, a voltage sensor and/or a magneto-electric sensor. Moreover, the coupling circuits may be designed to provide temperature control (i.e. heating, cooling and/or measurement of temperature) in a nearby location, e.g. a (bio-) chemical reaction chamber. In the aforementioned applications, it is often necessary to bring the coupling circuits as close as possible to an object. The proposed microchip allows such close contacting as the access to its sensitive front side is not hindered by bulky external connections.

According to a particular embodiment of the invention, the coupling circuits comprise circuits for the generation of an electromagnetic field, for example wires through which (AC or DC) currents can be directed to generate (alternating or static) magnetic fields. Additionally or alternatively, the coupling circuits may comprise circuits for the detection of an electromagnetic field, particularly a magnetic sensor device like a Giant Magneto Resistance (GMR) for the detection of magnetic fields. If both circuits for the generation and the detection of electromagnetic fields are provided, the microchip is especially apt for biosensor applications of the kind referred to above.

The invention further relates to microchip assembly comprising the following components:
a) A microchip of the kind described above, i.e. a microchip with a substrate, processing circuits and at least one recess in the front side of the substrate, and with terminal pads in said recesses.
b) A filling that at least partially embeds the microchip. The filling may particularly be a plastic or epoxy material or a glue. Moreover, the filling preferably embeds the microchip completely with the exception of the front side where the processing circuits are located.
c) Electrical tracks that are located on one surface of the filling (called “front side” of the filling in the following) in or below a plane that comprises the front side of the substrate/microchip and that are connected to the pads of the microchip. Note that the definition of “below” is such that the substrate is “below” its front side.

The microchip assembly described above has the advantage that the processing circuits in the front side of the microchip (being by definition identical to the front side of the substrate of the microchip) are very well accessible, because the electrical tracks that connect these circuits to external devices are located in or below said front side.

The invention further relates to a microfluidic device with at least one sample chamber in which liquid, gaseous or solid samples can be provided, particularly to a biosensor for the investigation of biological samples, which comprises a microchip of the kind described above. The microfluidic device may preferably comprise a microchip with coupling circuits for wireless physical interactions in the front side of a substrate. The free accessibility of the coupling circuits in the front side can be exploited in such a microfluidic device in various ways to improve the contact between the microchip and a sample in the sample chamber of the device.

The invention further relates to a process for the production of a microchip assembly of the kind described above, said process comprising the following steps:
a) Bonding the terminal pad(s) of a microchip of the kind described above (i.e. a microchip with a substrate, processing circuits and at least one recess in the front side of the substrate, and with pads in said recesses) to electrical tracks on the surface of a carrier substrate, wherein said carrier substrate may for example be a metal like copper or aluminum.
b) Embedding said microchip in a filling, wherein the processing circuits in the front side of the microchip are typically left free from the filling or at most covered by a very thin layer of filling material.
c) Removal of the carrier substrate, for example by etching, peeling or machining.

The aforementioned process has the advantage that electrical tracks with a precise geometry can be bonded to the microchip because they are initially fixed to a carrier substrate. After embedding the microchip in the filling and the attachment of the electrical tracks to said filling, the carrier substrate can be removed to provide a free access to the front side of the microchip.

According to further development of the process, a recess is produced in the carrier substrate before step a), wherein the processing circuits of the microchip and the associated substrate material can protrude into said recess during and after the bonding step a). This allows the production of a microchip assembly in which the front side of the microchip with the processing circuits assumes an elevated, completely accessible position.

These and other aspects of the invention will be apparent from and elucidated with reference to the embodiment(s) described hereinafter. Like reference numbers in the Figures (or numbers differing by ±10) refer to identical or similar components.

In the following the invention is described by way of example with the help of the accompanying drawings in which:

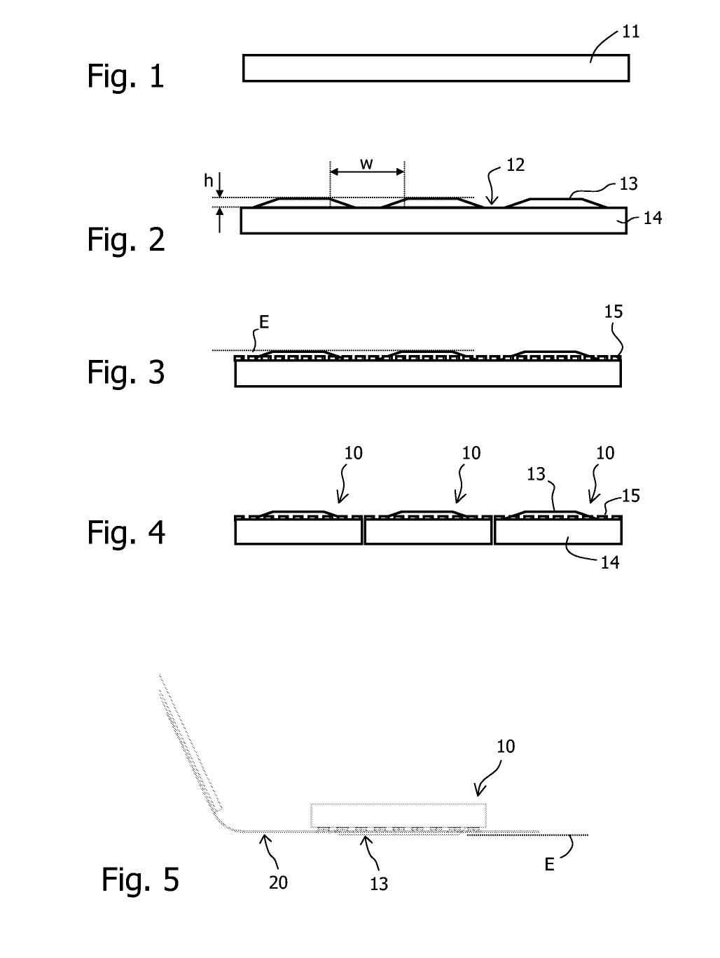

FIG. 1-5 show consecutive steps of the production of a microchip according to the present invention;

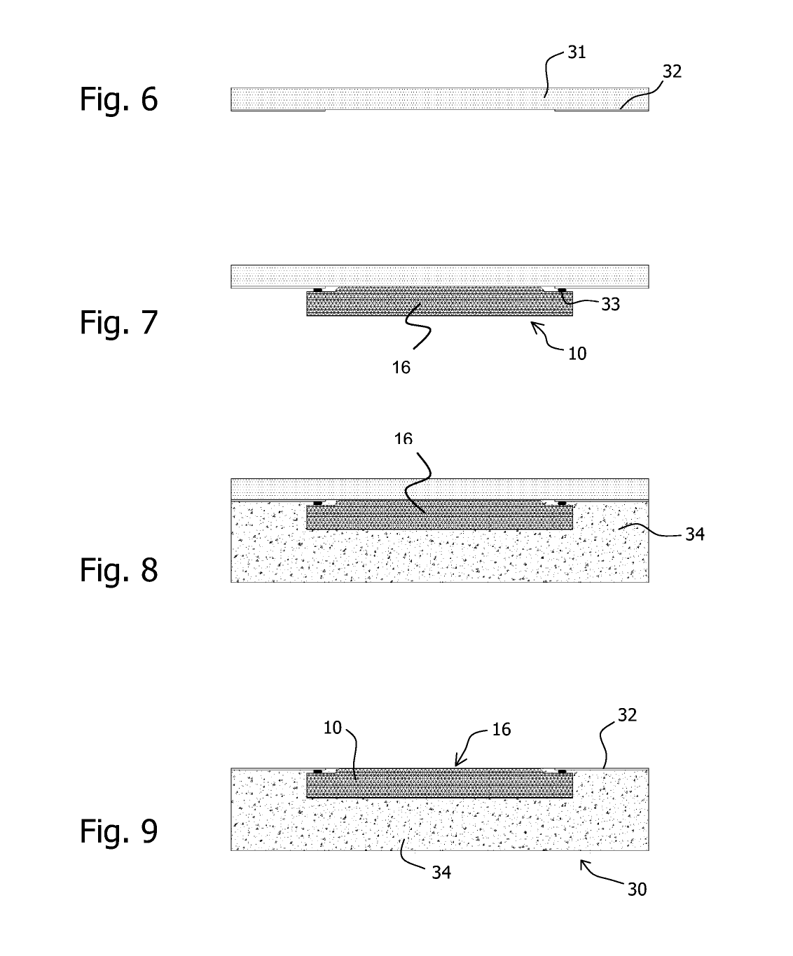

FIG. 6-9 show consecutive steps of the production of a first embodiment of a microchip assembly, wherein the sensitive surface of the microchip lies in the front side of the assembly;

FIG. 10-14 show consecutive steps of the production of a second embodiment of a microchip assembly, wherein the sensitive surface of the microchip lies above the front side of the assembly;

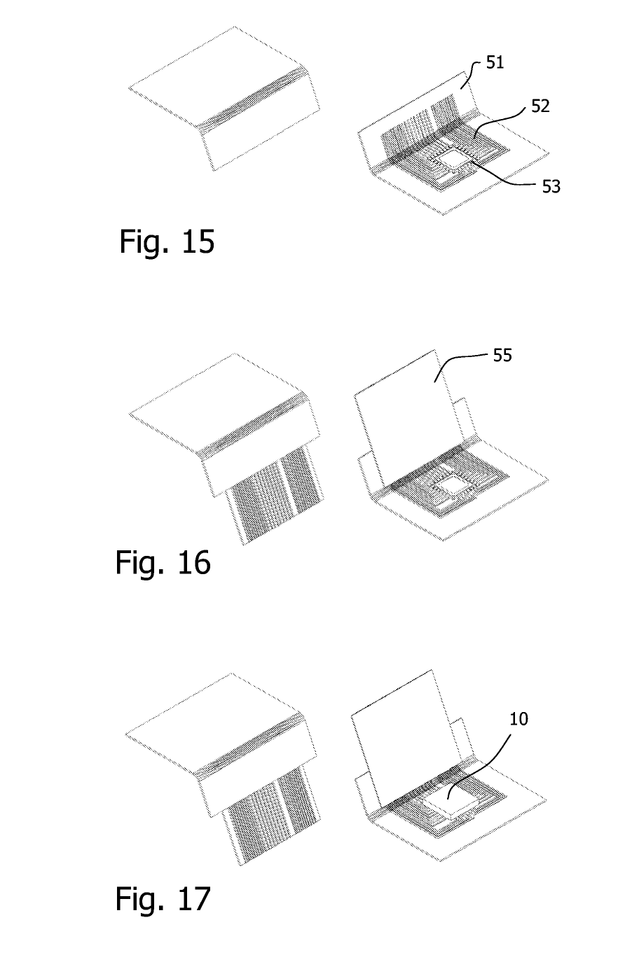

FIG. 15-20 show consecutive steps of the production of a third embodiment of a microchip assembly in perspective top (left) and bottom (right) views.

The devices described in the following may particularly be used for (magnetic) biosensors or biochips, though the invention is not limited thereto and can be applied to all sensors that require electrical connections, e.g. capacitive sensors, electronic light detectors, Ampere metric sensors, Volta metric sensors, magneto-electric sensors, etc. Moreover, such a device may be designed to provide temperature control in a sample space, for example if it is integrated into the (bottom) wall of a Polymerase Chain Reaction (PCR) chamber used for DNA amplification.

Magneto-resistive biochips have promising properties for bio-molecular diagnostics, in terms of sensitivity, specificity, integration, ease of use, and costs. Examples of such biochips are for example described in WO 2003/054566, WO 2003/054523, WO 2005/010542 A2, WO 2005/010543 A1 or Rife et al. (Sens. Act. A vol. 107, p. 209 (2003)), which are incorporated into the present application by reference. The known biosensors have however several drawbacks, namely:
Only a small amount of the main liquid flow will reach the sensor surface as it is located in a cavity of the sample chamber wall.
A large liquid flow is required to remove air bubbles from the cavity.
These two problems increase the amount of required antigens and magnetic beads, they complicate washing and fluid handling in the cartridge and they are not compatible with an in-expensive, easy to use and disposable biosensor.

The origin of these drawbacks is quite principally: the sensitive surface of the sensor and the sensor connections are located in the same plane. A solution for this problem is to contact the sensor at a plane, which is not its sensitive plane (“front side”). Several embodiments of this approach will be described in the following. These embodiments can particularly be used in a read-head device, which is brought in close contact to a carrier with magnetic beads. The carrier can for example be a foil, a microtiter well or a porous medium. Such a read-head sensor can be re-used several times on many different samples.

FIGS. 1-5 show five consecutive steps of the production of a microchip 10 according to the present invention, i.e. with an interconnect level below the chip surface or front plane E. This brings the sensitive surface of the chip at the level or even above the level of the chip package.

FIG. 1 schematically shows a side view of a raw substrate layer, e.g. a Si wafer 11. In FIG. 2, a rectangular grid of grooves or recesses 12 has been produced by dicing in the top surface of the wafer 11, resulting in a substrate 14 with isolated elevations of material on top. Instead of dicing, other suitable processes may be applied, for example etching with KOH or reactive ion etch (RIE). The top sides 13 of the elevations, called “front sides” of the substrate/microchip in the following, all lie in a “front plane” E. It should be noted that the “front plane” of the substrate denotes an abstract geometrical object with infinite extension, which comprises the aforementioned “front sides” of the substrate. A typical width w of the recesses 12 is 300 μm, while their depth h with respect to the front plane E is typically more than 30 μm.

After dicing of the recesses 12, metallization or other processing steps known to a person skilled in the art may be performed to produce the required processing circuits 16 on the front side 13 of the substrate. As was already mentioned, the processing circuits 16 may particularly be coupling circuits with electrical tracks for generating magnetic fields and sensing devices like a GMR (Giant Magnetic Resonator) for sensing magnetic fields. An important feature of the processing circuits 16 is that their terminal pads are located on the bottom sides of the recesses 12.

FIG. 3 shows the wafer after bumps 15 of soldering material, gold or the like have been deposited on the aforementioned pads. As known to a person skilled in the art, such bumps 15 may be used for bonding (e.g. flip-chip-bonding by soldering, gluing, ultrasonic bonding or the like) of external leads to electrically contact to the processing circuits 16. The bumps 15 are applied to terminal pads of processing circuits 16. The terminal pads connect the processing circuit 16 with electrical tracks. This means, terminal pads are mounted to the processing circuit 16. Note that the bumps 15 remain below the front side 13/front plane E of the substrate.

In FIG. 4, the wafer has been cut or separated into individual microchips 10, and FIG. 5 schematically shows how one of these microchips 10 is attached to a substrate 20 with tracks. It can be seen that the front side 13 with the processing circuits 16 extends beyond the interconnection plane.

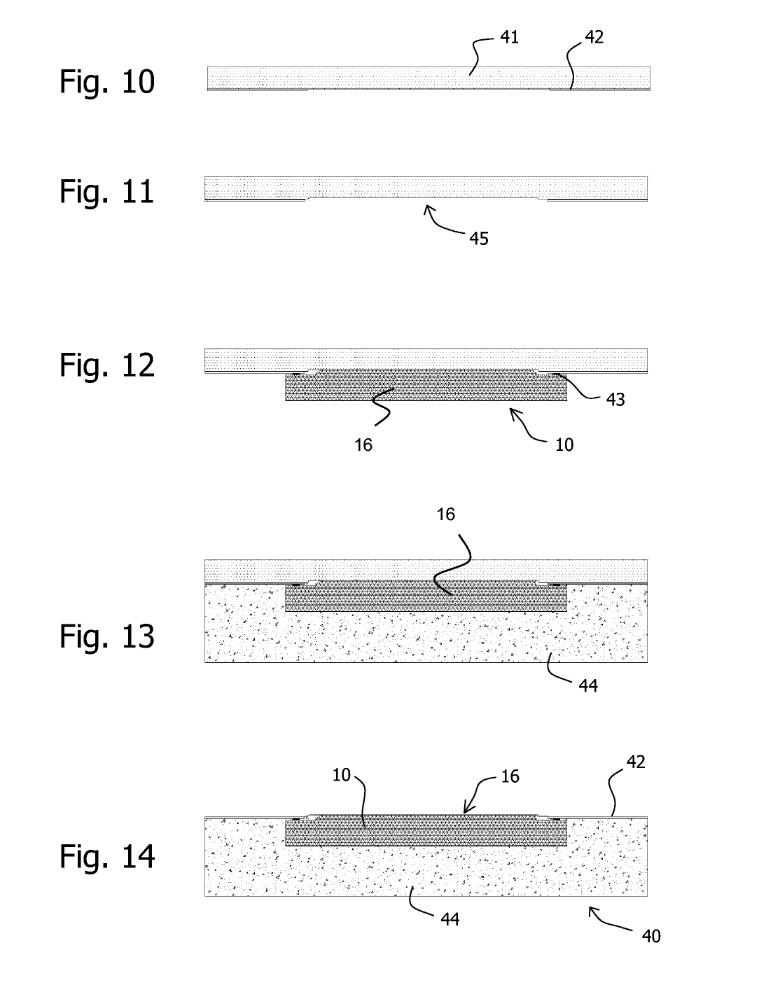

FIGS. 6-9 show in more detail four consecutive steps of the production of a microchip assembly 30 with a microchip 10 of the kind described above. The production starts in FIG. 6 with a carrier substrate 31 (e.g. a metal like Cu or Al) with electrical tracks 32 on its bottom side. In FIG. 7, the microchip 10 is bonded with bumps 33 to the electrical tracks 32 of the carrier substrate 31. FIG. 8 shows the microchip assembly after the microchip 10 has been embedded or overmolded in a filling 34. This filling may for example be a plastic or a glue. According to FIG. 9, the carrier substrate 31 may finally be removed, for example by etching or peeling, after the filling 34 has solidified. This results in a microchip assembly 30 with the sensitive side 13 of the microchip being in the front side of the assembly.

FIGS. 10-14 show consecutive steps of the production of an alternative microchip assembly 40. Components that are identical or a similar to those of the embodiment in FIGS. 6-9 are indicated with the same reference numbers plus 10.

FIG. 10 shows the initial carrier substrate 41 with electrical tracks 42 on its bottom side.

The main difference to the first production process of FIGS. 6-9 is illustrated in FIG. 11: the second production method is characterized in that a recess 45 is produced at the bottom side of the carrier substrate at locations not covered with the tracks 42 (methods to achieve this are for example described in the WO 2003/086037).

FIGS. 12-14 then show the bonding of a microchip 10 via bumps 43, its embedding in a filling 44, and the removal of the carrier layer to yield the final microchip assembly 40. These steps are similar to those shown in FIGS. 7-9 and need not be described in detail again. FIG. 14 shows, however, that the sensitive side 13 of the microchip now projects above the front plane of the assembly.

FIGS. 15-20 illustrate the production of a third microchip assembly 50. The Figures show the produced article in a perspective view from top on the left-hand side and from bottom on the right-hand side. For the sake of simplicity, only one device is drawn, while in practice an array of strips with multiple products is produced.

FIG. 15 shows a bended temporary metal carrier substrate 51 with conductor tracks 52 and contact pads 53 on one side. FIG. 16 shows how a flex foil 55 for the interconnection of the microchip assembly to external devices is attached to the temporary substrate 51 and its conductor tracks 52, respectively. In FIG. 17, a microchip 10 of the kind described above is bonded to the current conductors 52 via the corresponding bumps.

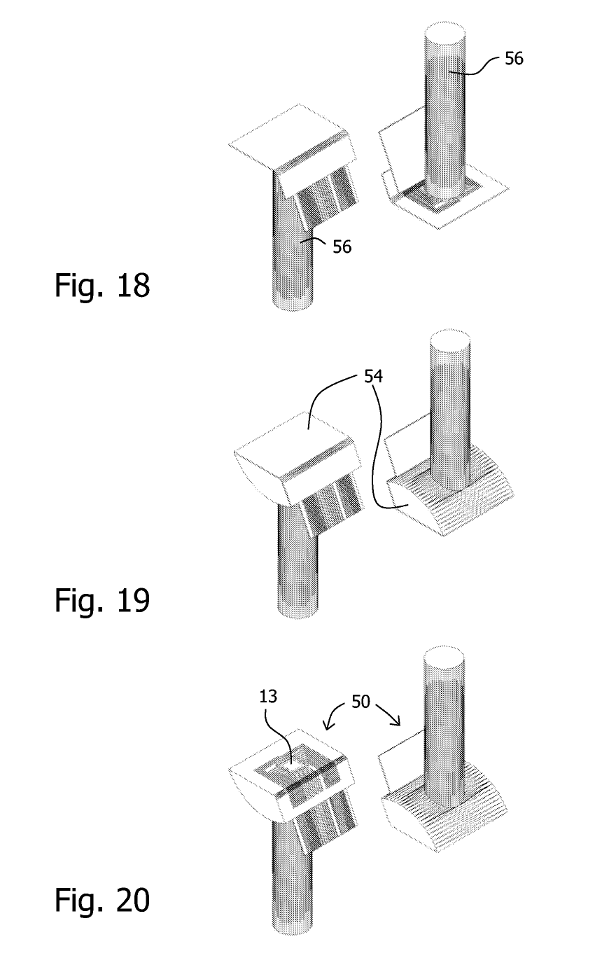

FIG. 18 shows an optional step, wherein a support pin 56 is attached (e.g. glued) to the microchip. In FIG. 19, an underfill material 54 is applied which embeds the microchip 10, the conductor tracks 52, and (partially) the support pin 56.

In the final step of FIG. 20, the temporary metal substrate 51 has been removed, resulting in a microchip assembly 50, wherein the sensitive front side 13 of the microchip lies in or above the plane of the whole package.

The sensor chip 10 of the embodiments described in the Figures has a typical area of 1.4×1.5 mm. It typically comprises 30 bondpads with a pitch of 150 μm, the thickness of the leads being about 10 μm, and the total thickness of the interconnection above the sensor surface being less than 30 μm.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. A microchip (10), comprising
a) a substrate (14);
b) processing circuits (16) in a front side (13) of the substrate (14);
c) at least one recess (12) in said front side (13) of the substrate (14) which has a depth (h) of more than 20 μm;
d) at least one terminal pad for electrical connections of external lines to the processing circuits (16), the terminal pad being located in the recess (12).

2. The microchip (10) according to claim 1, characterized in that the recess (12) has a depth (h) of more than 30 μm with respect to the front side (13).

3. The microchip (10) according to claim 1, characterized in that the recess (12) has a width (w) ranging from 100 μm to 1000 μm, preferably from 200 μm to 400 μm.

4. The microchip (10) according to claim 1, characterized in that a bump (15) is disposed on the pad which does not project beyond the front side (13) of the substrate (14).

5. The microchip (10) according to claim 1, characterized in that the processing circuits (16) comprise coupling circuits (17) that are adapted to perform and process a wireless physical interaction, the coupling circuits preferably implementing a temperature controller, a capacitive sensor, a light sensor, a current sensor, a voltage sensor and/or a magneto-electric sensor.

6. The microchip (10) according to claim 5, characterized in that the coupling circuits (17) comprise circuits for the generation of an electromagnetic field and/or circuits for the detection of an electromagnetic field, particularly a Giant Magneto Resistance.

7. A microchip assembly (30, 40, 50), comprising
a) a microchip (10) according to claim 1;
b) a filling (34, 44, 54) that embeds the microchip (10);
c) electrical tracks (32, 42, 52) that are located on the front side of the filling (34, 44, 54) in or below a plane (E) comprising the front side (13) of the microchip's substrate and that are connected to the terminal pads of the microchip (10).

8. A microfluidic device with a sample chamber, particularly a biosensor, comprising a microchip (10) according to claim 1.

9. A process for the production of a microchip assembly (30, 40, 50) according to claim 7, comprising the following steps:
a) bonding the at least one terminal pad of an associated microchip (10) to electrical tracks (32, 42, 52) on the surface of a carrier substrate (14);
b) embedding said microchip (10) in a filling (34, 44, 54);
c) removing the carrier substrate (14).

10. The process according to claim 9, characterized in that a recess (45) is produced in the carrier substrate (14) into which the processing circuits (16) of the microchip (10) can protrude after the bonding of step a).
